(19) United States
(12) Patent Application Publication
(10) Pub. No.: US 2007/0135847 A1
(43) Pub. Date: Jun. 14, 2007

(54) Subcutaneous defibrillation system and method using same

(76) Inventor: Bruce H. Kenknight, Maple Grove, MN (US)

Correspondence Address: HOLLINGSWORTH & FUNK, LLC, Suite 125, 8009 34th Avenue South, Minneapolis, MN, 55425, US

(21) Appl. No.: 11/301,241

(22) Filed: Dec. 12, 2005

(51) Int. Cl. A61N 1/39

(52) U.S. Cl. 607/5

(57) ABSTRACT

A medical system includes a housing configured for implantation within a patient's subclavicular region. Detection circuitry provided in the housing is configured to detect cardiac electrical activity. Energy delivery circuitry provided in the housing is configured to deliver therapy to treat a detected tachycardia or fibrillation episode. A lead is coupled to the housing, detection circuitry, and energy delivery circuitry. The lead is configured for subcutaneous, non-intrathoracic placement within the patient, and extends from the subclavicular region to just below the patient's ribs or the subxiphoid process. A defibrillation electrode is provided at a distal end of the lead body. A pair of sensing electrodes is provided on the lead body at locations consistent with positions V2-V5 of surface electrocardiogram electrodes.

### FIELD OF THE INVENTION

The present invention relates generally to implantable medical devices and, more particularly, to subcutaneous sensing and defibrillation devices, and methods using same.

### BACKGROUND OF THE INVENTION

The healthy heart produces regular, synchronized contractions. Rhythmic contractions of the heart are normally controlled by the sinoatrial (SA) node, which are specialized cells located in the upper right atrium. The SA node is the normal pacemaker of the heart, typically initiating 60-100 heart beats per minute. When the SA node is pacing the heart normally, the heart is said to be in normal sinus rhythm.

If the heart's electrical activity becomes uncoordinated or irregular, the heart is denoted to be arrhythmic. Cardiac arrhythmia impairs cardiac efficiency and can be a potential life threatening event. Cardiac arrhythmias have a number of etiological sources, including tissue damage due to myocardial infarction, infection, or degradation of the heart's ability to generate or synchronize the electrical impulses that coordinate contractions.

Bradycardia occurs when the heart rhythm is too slow. This condition may be caused, for example, by impaired function of the SA node, denoted sick sinus syndrome, or by delayed propagation or blockage of the electrical impulse between the atria and ventricles. Bradycardia produces a heart rate that is too slow to maintain adequate circulation.

When the heart rate is too rapid, the condition is denoted tachycardia. Tachycardia may have its origin in either the atria or the ventricles. Tachycardias occurring in the atria of the heart, for example, include atrial fibrillation and atrial flutter. Both conditions are characterized by rapid contractions of the atria. Besides being hemodynamically inefficient, the rapid contractions of the atria can also adversely affect the ventricular rate.

Ventricular tachycardia occurs, for example, when electrical activity arises in the ventricular myocardium at a rate more rapid than the normal sinus rhythm. Ventricular tachycardia can quickly degenerate into ventricular fibrillation. Ventricular fibrillation is a condition denoted by extremely rapid, uncoordinated electrical activity within the ventricular tissue. The rapid and erratic excitation of the ventricular tissue prevents synchronized contractions and impairs the heart's ability to effectively pump blood to the body, which is a fatal condition unless the heart is returned to sinus rhythm within a few minutes.

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses which are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating the arrhythmias described above.

Implantable cardioverter/defibrillators (ICDs) have been used as an effective treatment for patients with serious cardiac arrhythmias. For example, a typical ICD includes one or more endocardial leads to which at least one defibrillation electrode is connected. Such ICDs are capable of delivering high energy shocks to the heart, interrupting the ventricular tachyarrhythmia or ventricular fibrillation, and allowing the heart to resume normal sinus rhythm.

### SUMMARY OF THE INVENTION

The present invention is directed to cardiac sensing and stimulation systems and methods. Embodiments of the present invention include those directed to subcutaneous cardiac stimulation methods and systems that detect and treat cardiac arrhythmia.

According to one embodiment, a medical system includes a housing configured for implantation within a patient's subclavicular region. Detection circuitry is provided in the housing and configured to detect cardiac electrical activity. Energy delivery circuitry is provided in the housing and configured to deliver therapy to treat a detected tachycardia or fibrillation episode. A lead is coupled to the housing, detection circuitry, and energy delivery circuitry. The lead is configured for subcutaneous, non-intrathoracic placement within the patient.

The lead comprises a lead body configured to extend from the patient's subclavicular region to a location just below the patient's ribs or the subxiphoid process of the patient's sternum. A defibrillation electrode is provided at a distal end of the lead body. A pair of sensing electrodes is provided on the lead body at locations that overlie a lateral aspect of the patient's left ventricle between the third and eleventh ribs. For example, the pair of sensing electrodes is provided on the lead body at locations consistent with positions V2-V5 of surface electrocardiogram electrodes.

The pair of sensing electrodes may comprise ring electrodes. The defibrillation electrode may comprise a coil electrode or a screen patch electrode, for example.

Communications circuitry may be provided in the housing. The communications circuitry is configured to facilitate communications between the implanted system and a patient-external device. For example, the communications circuitry may be configured to facilitate communications between the system and a hand-held or bedside communications device. By way of further example, the communications circuitry may be configured to facilitate communications between the system and an interface of a network. The network may be configured to support a patient management system.

The housing may be configured for implantation in the patient's subclavian region in closer proximity to a left axillary region than a sternal region of the patient. In one approach, the lead body is configured to extend from the patient's subclavicular region to a location just below the patient's ribs. In another approach, the lead body is configured to extend from the patient's subclavicular region to the subxiphoid process of the patient's sternum.

The energy delivery circuitry is preferably configured to deliver therapy to treat a detected tachycardia or fibrillation episode using a vector defined between the housing electrode and the defibrillation electrode. The detection circuitry is preferably configured to sense the cardiac electrical activity using a vector defined between the sense electrodes. The detection circuitry may be configured to sense the cardiac electrical activity using a vector defined between at least one of the sense electrodes and the housing electrode.

The housing electrode may comprise all or a portion of an electrically conductive enclosure of the housing. In another configuration, the housing may comprise a header, and the housing electrode may be supported by the header. In a further configuration, the housing electrode may be supported by a stub lead coupled to the housing. The stub lead may be configured to extend from the housing into the patient's left axilla and oriented posteriorly.

According to another embodiment, a method involves providing a pulse generator disposed in a housing and implanted within a patient's subclavicular region, and providing a lead coupled to the pulse generator and configured for subcutaneous, non-intrathoracic placement within the patient. The lead comprises a lead body configured to extend from the patient's subclavicular region to a location just below the patient's ribs or the subxiphoid process of the patient's sternum, a defibrillation electrode provided at a distal end of the lead body, and a pair of sensing electrodes provided on the lead body at locations that overlie a lateral aspect of the patient's left ventricle between the third and eleventh ribs.

The method further involves sensing cardiac electrical activity and detecting a tachycardia or fibrillation episode using a vector defined between the pair of sensing electrodes. The method also involves delivering a therapy to treat the detected tachycardia or fibrillation episode using a vector defined between the housing of the pulse generator and the defibrillation electrode.

Information concerning one or both of the cardiac electrical activity and the tachycardia or fibrillation episode may be stored within the housing. The information concerning one or both of the cardiac electrical activity and the tachycardia or fibrillation episode may be communicated to a patient-external device. For example, this information may be communicated to a network, such as one that supports a patient management system.

The method may further involve communicating information concerning one or both of the cardiac electrical activity and the tachycardia or fibrillation episode to an external device. Information concerning one or both of the cardiac electrical activity and the tachycardia or fibrillation episode may be analyzed for a variety of purposes. Such purposes may include assessing the patient's well-being, implementing or adjusting a therapy deliverable to the patient, generating a therapeutic recommendation for managing cardiac arrhythmia for the patient based at least in part on the analyzed information, or generating visual and/or aural output based at least in part on the analyzed information.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 shows a configuration of a transthoracic cardiac sensing/stimulation device implanted in the left chest region of a patient in accordance with an embodiment of the present invention;

FIG. 2 shows another configuration of a transthoracic cardiac sensing/stimulation device implanted in the left chest region of a patient in accordance with an embodiment of the present invention;

FIG. 3 shows a further configuration of a transthoracic cardiac sensing/stimulation device implanted in the left chest region of a patient in accordance with an embodiment of the present invention;

FIG. 4 is a block diagram showing various components of a transthoracic cardiac sensing/stimulation device in accordance with an embodiment of the present invention; and

FIG. 5-7 illustrate various methods involving use or implantation of a transthoracic cardiac sensing/stimulation device of the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings which form a part hereof, and in which is shown by way of illustration, various embodiments in which the invention may be practiced. It is to be understood that other embodiments may be utilized, and structural and functional changes may be made without departing from the scope of the present invention.

An implanted device of the present invention can include one or more of the features, structures, methods, or combinations thereof described hereinbelow. For example, a cardiac stimulation device can be implemented to include one or more of the advantageous features and/or processes described below. It is intended that such a device need not include all of the features described herein, but can be implemented to include selected features that provide for useful structures and/or functionality. Such a device may be implemented to provide a variety of therapeutic or diagnostic functions. One such device, termed an implantable transthoracic cardiac sensing/stimulation (ITCS) device, is described herein to include various advantageous features and/or processes. It is understood that the description of features and processes within the context of an ITCS device is provided for non-limiting illustrative purposes only.

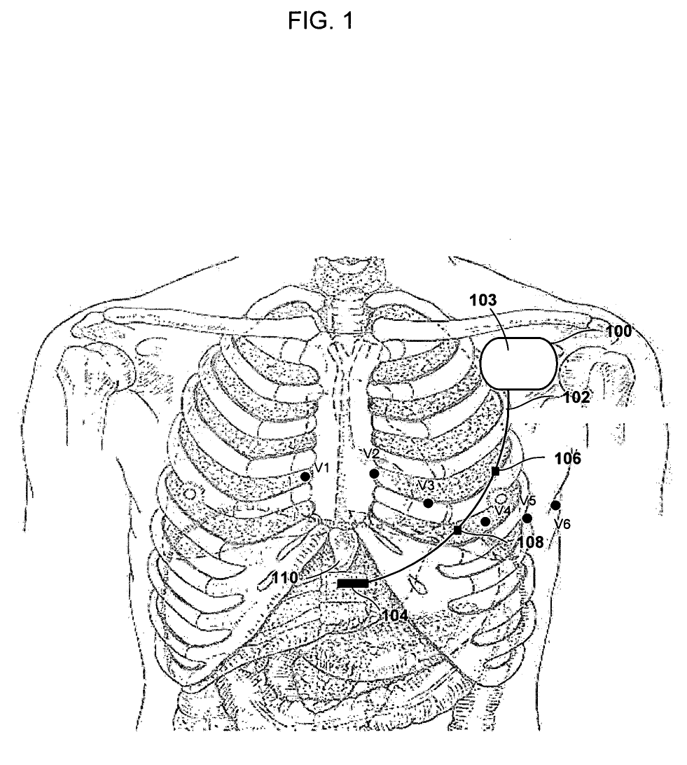

Referring now to FIGS. 1-3 of the drawings, there is shown various configurations of an ITCS device implanted in the left chest region of a patient. In the particular configurations shown in FIGS. 1-3, the ITCS device includes a housing 100 within which various cardiac sensing, detection, processing, and energy delivery circuitry can be housed. Communications circuitry is disposed within the housing 100 for facilitating communication between the ITCS device and an external communication device, such as a portable or bed-side communication station, patient-carried/worn communication station, external programmer, or network interface of an advanced patient management system, for example. The housing 100 is typically configured to include at least one electrode, such as a can electrode, an indifferent electrode provided in a header of the housing 100, or an electrode provided on a short stub lead extending from the header of the housing 100. A lead 102 extends from the housing 100 and generally along the lateral aspect of the left chest to a location particularly well-suited for sensing cardiac electrical activity and delivering therapy to the heart, as is described in greater detail below.

According to the configuration shown in FIG. 1, the housing 100 of the ITCS device is shown implanted in the subclavicular region of the patient. The housing 100 is typically implanted via a subclavicular pocket created on the anterior chest wall. The housing 100 is preferably positioned to be closer to the axillary region than the sternal region. In the configuration shown in FIG. 1, the housing 100 is configured as an active can, such that the housing 100 includes a conductive enclosure 103 that may encompass all or a portion of housing 100.

A single lead 102 is coupled to the housing 100 and extends to a location proximate the subxyphoid process 110 of the sternum. Lead 102 includes a defibrillation electrode 104 provided at a distal end of lead 102. Defibrillation electrode 104 is typically a coil electrode, but may be of a different configuration. Useful defibrillation electrode configurations include multi-element coils, spiral coils, spiral coils mounted on non-conductive backing, and screen patch electrodes, for example. Defibrillation electrode 104 may be configured to assume a variety of shapes, and comprise one or multiple electrode elements, such as an array or band of electrodes.

Lead 102 further includes a pair of sensing electrodes 106, 108 disposed on the body of the lead at locations proximal of the defibrillation electrode 104. The sensing electrodes 106, 108 are preferably ring electrodes, but may be of a different configuration.

The position of sensing electrodes 106, 108 on the lead 102 is predefined based on a number of factors. Such factors include, for example, a typical patient's thoracic dimensions (e.g., chest size), the target implantation location of the housing, and the target implantation location of the defibrillation electrode 104. The length of lead 102 and/or positioning of sensing electrodes 106, 108 on the lead 102 may vary based on these and other factors, such as the patient's age, gender, size, etc.

FIG. 1 shows typical surface electrocardiogram (ECG) electrode positions appropriate for the particular patient's chest depicted in FIG. 1. In typical use, surface ECG electrodes V1, V2, V3, V4, V5, and V6 are used to assess the electrical activity of the heart in the horizontal plane; i.e., as if looking down on a cross section of the body at the level of the heart. The surface ECG electrode locations shown in FIG. 1 are as follows:

V1: Positioned in the 4th intercostal space just to the right of the sternum.

V2: Positioned in the 4th intercostal space just to the left of the sternum.

V3: Positioned halfway between V2 and V4.

V4: Positioned at the 5th intercostal space in the mid-clavicular line.

V5: Positioned in the anterior axillary line at the same level as V4.

V6: Positioned in the mid axillary line at the same level as V4 and V5.

Surface ECG electrodes at positions V1 and V2 are typically used to monitor electrical activity of the heart from the anterior aspect, septum, and right ventricle. Surface ECG electrodes at positions V3 and V4 are typically use to monitor electrical activity of the heart from the anterior aspect. Surface ECG electrodes at positions V5 and V6 are typically used to monitor electrical activity of the heart from the left ventricle and lateral aspect.

A factor that may influence the position of the sensing electrodes 106, 108 on lead 102 relative to the housing location involves the location of sensing electrodes 106, 108 in relation to surface ECG electrode locations applicable for the patient. In general, sensing electrodes 106, 108 are preferably disposed on the body of lead 102 so that sensing electrodes 106, 108 are positioned at locations that overlie a lateral aspect of the patient's left ventricle between the third and eleventh ribs. More particularly, the sensing electrodes 106, 108 are preferably disposed on the body of lead 102 at locations consistent with positions V2-V5 of surface ECG electrodes.

The sensing electrodes 106, 108 are used to sense electrical activity of the heart. Sensing electrodes 106, 108 are typically used in a bipolar sensing mode. Alternatively, one of the sensing electrodes 106, 108, together with the housing electrode 103, may be used in a unipolar sensing mode.

Various therapies are delivered to the heart via a vector defined between the defibrillation electrode 104 and the housing electrode 103. Typical therapies deliverable to the heart include cardioversion, defibrillation, and anti-tachycardia pacing therapies. It is noted that lower energy therapies, such as anti-tachycardia pacing therapies, may use an energy delivery vector that implicates one or both of the sensing electrodes 106, 108, and may include or exclude defibrillation electrode 104.

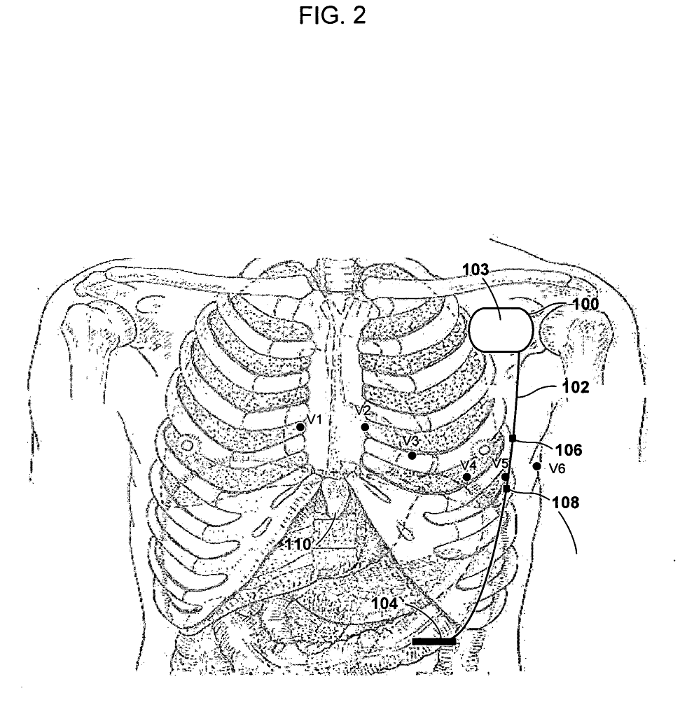

In the configuration shown in FIG. 2, the lead 102 is configured to extend from the housing 100 (located in the left subclavicular region) to a location just below the patient's ribs (i.e., just below the patient's left false ribs). In particular, the lead 102 is configured to extend from the housing 100 and is of sufficient length so that defibrillation electrode 104 is positioned at a location just below the patient's ribs of the left chest. In addition, sensing electrodes 106, 108 are preferably disposed on the body of lead 102 at locations consistent with positions V2-V5 of surface ECG electrodes.

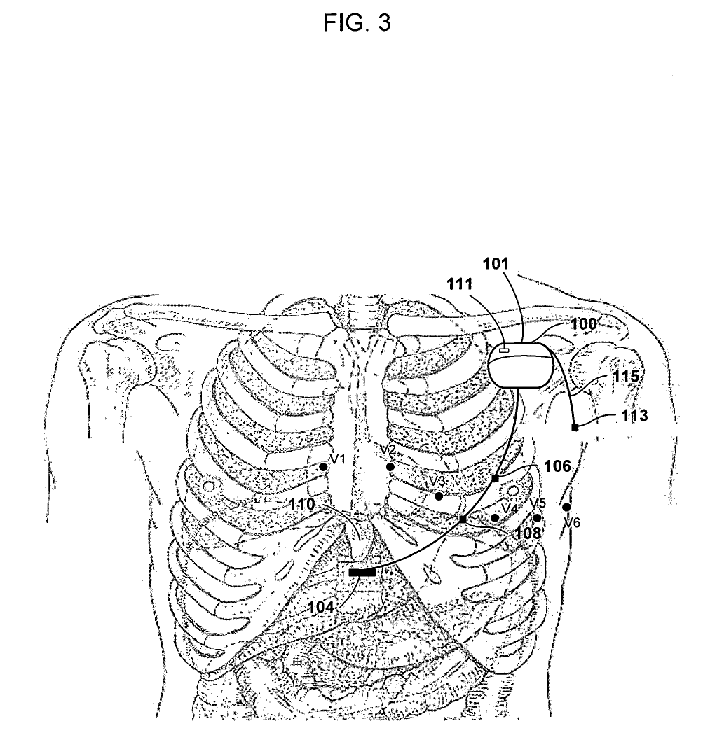

FIG. 3 shows another embodiment of an ITCS device of the present invention. The ITCS device of FIG. 3 has a general configuration equivalent to that shown in FIG. 1. FIG. 3 shows alternative housing electrode configurations. In one configuration, housing 100 may include a header 101 which supports an indifferent electrode 111. In another configuration, a stub lead 115 extends from the header 101 of the housing 100. An electrode 113 is disposed at a distal end of the stub lead 115. The stub lead 115 is preferably directed toward the patient's left axilla and angling posteriorly. It is understood that the housing 100 need only include one electrode (e.g., can, indifferent, stub lead electrode), but may be configured to include more than one electrode of same or different configuration.

Lead 102 may be of a conventional design and constructed using conventional materials. Alternatively, lead 102 may be constructed to be somewhat flexible, yet has an elastic, spring, or mechanical memory that retains a desired configuration after being shaped or manipulated by a clinician. For example, lead 102 can incorporate a gooseneck or braid system that can be distorted under manual force to take on a desired shape. In this manner, lead 102 can be shape-fit to accommodate the unique anatomical configuration of a given patient, and generally retains a customized shape after implantation.

Lead 102 may alternatively include a rigid elongated structure that positionally stabilizes the subcutaneous electrodes with respect to the housing 100. In this configuration, the rigidity of the elongated structure maintains a desired spacing between the subcutaneous electrodes and the housing 100, and a desired orientation of the subcutaneous electrodes/housing relative to the patient's heart. The elongated structure can be formed from a structural plastic, composite or metallic material, and comprises, or is covered by, a biocompatible material. Appropriate electrical isolation between the housing 100 and subcutaneous electrodes is provided in cases where the elongated structure is formed from an electrically conductive material, such as metal.

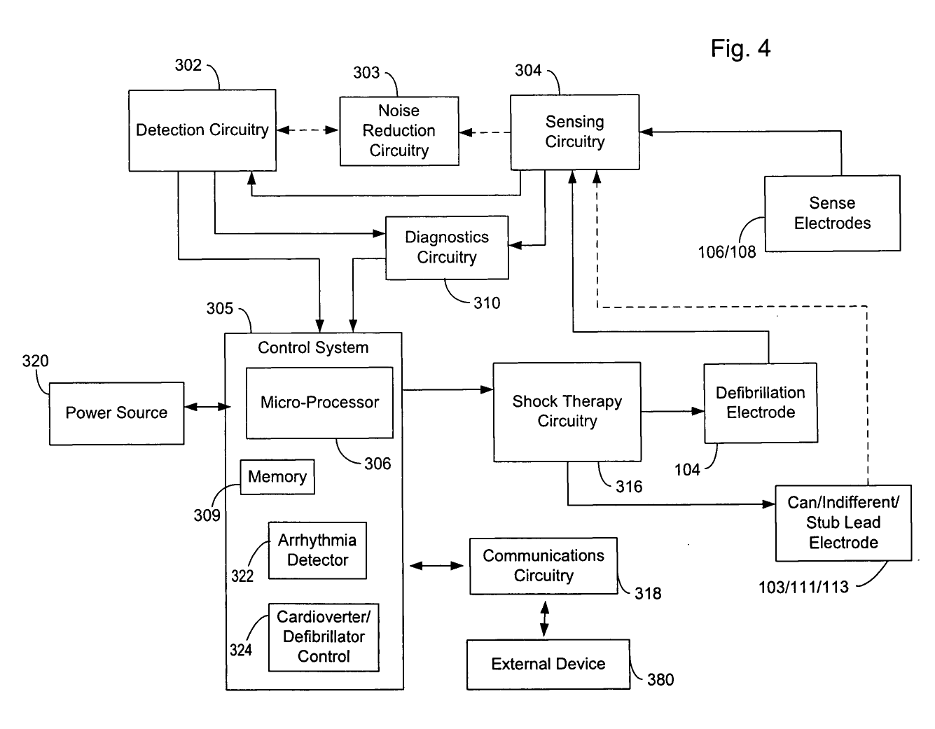

FIG. 4 is a block diagram depicting various components of an ITCS device in accordance with one embodiment of the present invention. It is understood that various components and functionality depicted in FIG. 4 and described herein can be implemented in hardware, software, or a combination of hardware and software. It is further understood that the components and functionality depicted as separate or discrete blocks/elements can be implemented in combination with other components and functionality, and that the depiction of such components and functionality in individual or integral form is for purposes of clarity of explanation, and not of limitation.

According to the embodiment shown in FIG. 4, the ITCS device incorporates a processor-based control system 305 which includes a micro-processor 306 coupled to appropriate memory (volatile and non-volatile) 309, it being understood that any logic-based control architecture can be used. The control system 305 is coupled to circuitry and components to sense, detect, and analyze electrical signals produced by the heart and deliver electrical stimulation energy to the heart under predetermined conditions to treat cardiac arrhythmias. The electrical energy delivered by the ITCS device may be in the form of lower energy pulses associated with anti-tachycardia pacing therapies or high energy pulses associated with cardioversion or defibrillation therapies.

Cardiac signals are sensed using the subcutaneous sensing electrodes 106, 108. Alternatively, at least one of the sensing electrodes 106, 108 (or defibrillation electrode 104) and at least one of the can 103, indifferent electrode 111, and stub lead electrode 113 may be used for far-field sensing of cardiac signals. Combinations of these electrodes may also be used to define a variety of useful sensing vectors. As such, unipolar and/or bipolar electrode configurations may be employed.

Sensed cardiac signals are received by sensing circuitry 304, which includes sense amplification circuitry and may also include filtering circuitry and an analog-to-digital (A/D) converter. The sensed cardiac signals processed by the sensing circuitry 304 may be received by noise reduction circuitry 303, which can further reduce noise before signals are sent to the detection circuitry 302. Noise reduction circuitry 303 may also be incorporated after detection circuitry 302 in cases where high power or computationally intensive noise reduction algorithms are required, such as source separation algorithms. Noise reduction circuitry 203 operates to improve the signal-to-noise ratio of sensed cardiac signals by removing noise content of the sensed cardiac signals introduced from various sources. Typical types of transthoracic cardiac signal noise includes electrical noise and noise produced from skeletal muscles, for example. A number of methodologies may be implemented to improve the signal-to-noise ratio of sensed cardiac signals in the presence of skeletal muscular induced noise and noise from other sources.

An arrhythmia detector 322 is shown as part of control system 305, but may optionally be incorporated in detection circuitry 302. Detection circuitry 302 typically includes a signal processor that coordinates analysis of the sensed cardiac signals and/or other sensor inputs to detect cardiac arrhythmias, such as, in particular, tachycardia and fibrillation. Detection and verification of arrhythmias can be accomplished using rate-based discrimination algorithms as known in the art implemented by arrhythmia detector 322. Arrhythmic episodes can also be detected and verified by morphology-based analysis of sensed cardiac signals as is known in the art. Tiered or parallel arrhythmia discrimination algorithms can also be implemented using both rate-based and morphologic-based approaches. Further, a rate and pattern-based arrhythmia detection and discrimination approach may be employed to detect and/or verify arrhythmic episodes.

Detection circuitry 302 communicates cardiac signal information to the control system 305. Memory circuitry 309 of the control system 305 contains parameters for operating in various sensing, defibrillation, and diagnostic modes, and stores data indicative of cardiac signals received by the detection circuitry 302. The memory circuitry 309 can also be configured to store historical ECG and therapy data, which may be used for various purposes and transmitted to an external receiving device 380 as needed or desired via communications circuitry 318.

According to a configuration that provides cardioversion and defibrillation therapies, the control system 305 processes cardiac signal data received from the detection circuitry 302 and initiates appropriate tachyarrhythmia therapies to terminate cardiac arrhythmic episodes and return the heart to normal sinus rhythm. The cardioverter/defibrillation control 324 of control system 305 is coupled to shock therapy circuitry 316. The shock therapy circuitry 316 is coupled to defibrillation electrode 104 and one of the can 103, indifferent electrode 111 or stub lead electrode 113. A switch matrix may be implemented to select various sense vectors and therapy delivery vectors.

Upon command, the shock therapy circuitry 316 delivers cardioversion and defibrillation stimulation energy to the heart in accordance with a selected cardioversion or defibrillation therapy. In a less sophisticated configuration, the shock therapy circuitry 316 is controlled to deliver defibrillation therapies, in contrast to a configuration that provides for delivery of both cardioversion and defibrillation therapies. Shock therapy circuitry 316 may also be controlled to deliver anti-tachycardia pacing therapies. Exemplary ICD high energy delivery circuitry, structures and functionality, aspects of which can be incorporated in an ITCS device of a type contemplated herein, are disclosed in commonly owned U.S. Pat. Nos. 5,372,606; 5,411,525; 5,468,254; and 5,634,938, which are hereby incorporated herein by reference in their respective entireties.

Communications circuitry 318 is coupled to the micro-processor 306 of the control system 305. The communications circuitry 318 allows the ITCS device to communicate with one or more receiving devices or systems 380 situated external to the ITCS device. By way of example, the ITCS device can communicate with a patient-worn, portable or bed-side communication system, a programmer, an interface of a network server or other external device via the communications circuitry 318.

For example, an ITCS device of the present invention may be used within the structure of an advanced patient management (APM) system. The advanced patient management system allows physicians to remotely and automatically monitor cardiac and respiratory functions, as well as other patient conditions. In one example, an ITCS device may be equipped with various telecommunications and information technologies that enable real-time data collection, diagnosis, and treatment of the patient. Various ITCS embodiments described herein may be used in connection with advanced patient management. Methods, structures, and/or techniques described herein, which may be adapted to provide for remote patient/device monitoring, diagnosis, therapy, or other APM related methodologies, may incorporate features of one or more of the following references: U.S. Pat. Nos. 6,221,011; 6,270,457; 6,277,072; 6,280,380; 6,312,378; 6,336,903; 6,358,203; 6,368,284; 6,398,728; and 6,440,066, which are hereby incorporated herein by reference.

The communications circuitry 318 can allow the ITCS device to communicate with an external programmer. In one configuration, the communications circuitry 318 and the programmer unit use a wire loop antenna and a radio frequency telemetric link, as is known in the art, to receive and transmit signals and data between the programmer unit and communications circuitry 318. In this manner, programming commands and data are transferred between the ITCS device and the programmer unit during and after implant. Using a programmer, a physician is able to set or modify various parameters used by the ITCS device. For example, a physician can set or modify parameters affecting sensing, detection, and defibrillation functions of the ITCS device, including cardioversion/defibrillation therapy modes.

Typically, the ITCS device is encased and hermetically sealed in a housing suitable for implanting in a human body as is known in the art. Power to the ITCS device is supplied by an electrochemical power source 320 housed within the ITCS device.

Depending on the configuration of a particular ITCS device, a delivery system can advantageously be used to facilitate proper placement and orientation of the ITCS device housing and subcutaneous lead/electrodes. According to one configuration of such a delivery system, a long metal rod similar to conventional trocars can be used to perform small diameter blunt tissue dissection of the subdermal layers. This tool may be pre-formed straight or curved and be sufficiently flexible to facilitate placement of the subcutaneous electrode, or it may be flexible enough to allow the physician to shape it appropriately for a given patient. Such a delivery tool may include a coupling or grasping arrangement that engages the lead body and/or defibrillation electrode. In this configuration, the delivery tool guides the lead through a subcutaneous tunnel created by the tool. Exemplary delivery tools, aspects of which can be incorporated into an ITCS device delivery tool, are disclosed in commonly owned U.S. Pat. No. 5,300,106 and U.S. Publication Nos. 2004/0204735 and 2004/0204734, which are hereby incorporated herein by reference.

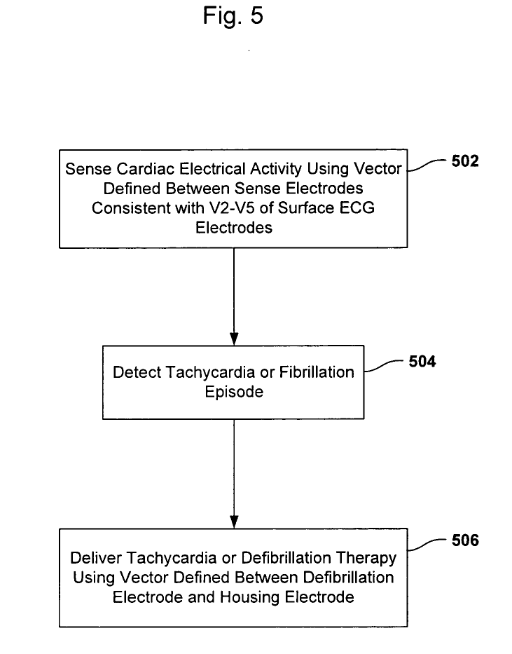

Turning now to FIG. 5-7, there is shown various methods involving use or implantation of an ITCS device of the present invention. FIG. 5 illustrates various processes associated with sensing of cardiac electrical activity and therapy delivery using an ITCS device. According to FIG. 5, cardiac electrical activity is sensed 502 using a vector defined between sense electrodes of a lead consistent with V2-V5 of surface ECG electrodes. A tachycardia or fibrillation episode is detected 504 and verified by the ITCS device. Appropriate tachycardia or defibrillation therapy is delivered 506 using a vector defined between the defibrillation electrode and housing electrode of the ITCS device.

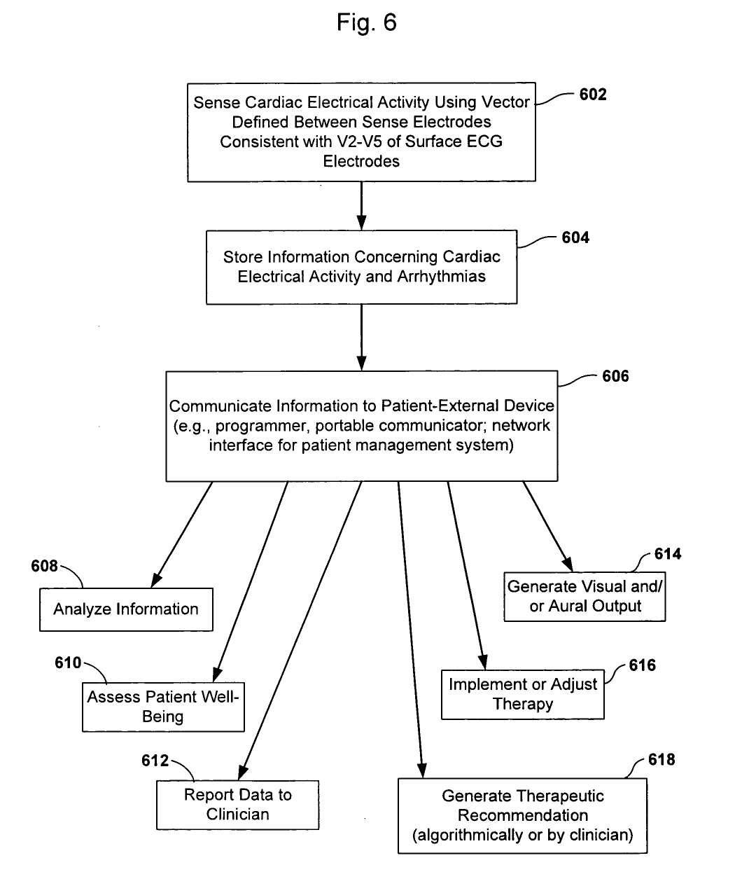

According to FIG. 6, cardiac electrical activity is sensed 602 using a vector defined between sense electrodes of the lead consistent with V2-V5 of surface ECG electrodes. Information concerning the sensed cardiac electrical activity and any arrhythmias is stored 604. This information is communicated 606 to a patient-external device, such as a programmer, portable communicator, or network interface of a patient management system. The information communicated from the ITCS device to the patient-external device may be used for a variety of purposes.

For example, the information may be analyzed 608 for a variety of purposes, such as to assess 610 patient well-being. One or more therapies may be implemented or adjusted 616 based on the information. The information and related data may be reported to a clinician 612, such as via a networked patient management system. Therapeutic recommendations may be generated 618 algorithmically or by a clinician using the information. Various forms of visual and aural output may be generated, including electronic and paper-based reports and data.

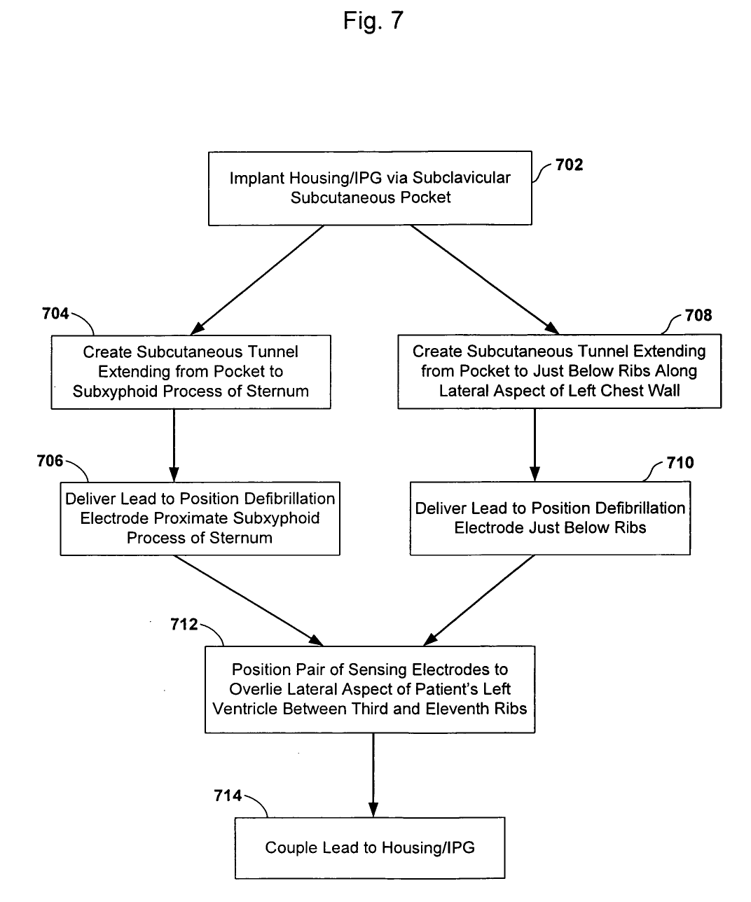

FIG. 7 shows various steps of a procedure for implanting an ITCS device in accordance with an embodiment of the present invention. According to FIG. 7, a housing/IPG is implanted 702 in a patient via a subclavicular subcutaneous pocket. According to one approach, a subcutaneous tunnel is created 704 that extends from the pocket to the subxiphoid process of the patient's sternum. The tunneling tool may be a preformed, flexible tool that also guides the lead body, and may include features of the tunneling tools described above. The lead is delivered 706 so as to position the defibrillation electrode proximate the subxiphoid process. The pair of sensing electrodes provided on the lead is positioned 712 to overlie a lateral aspect of the patient's left ventricle between the third and eleventh rib.

In alternative approach, a subcutaneous tunnel is created 708 that extends from the pocket to a location just below the ribs along a lateral aspect of the left chest wall. The lead is delivered 710 so as to position the defibrillation electrode at a location just below the ribs (e.g., false ribs of the left chest). The pair of sensing electrodes provided on the lead is positioned 712 to overlie a lateral aspect of the patient's left ventricle between the third and eleventh rib.

The tunneling tool is removed from the patient, and the lead is coupled 714 to the housing/IPG. In the case of a unitary ITCS, the pocket may first be formed, followed by creation of the subcutaneous tunnel. The lead may be guided through the tunnel and positioned at the desired location, followed by implantation of the housing/IPG.

An ITCS device of the present invention can incorporate circuitry, structures and functionality of the subcutaneous implantable medical devices disclosed in commonly owned U.S. Pat. Nos. 5,203,348; 5,230,337; 5,360,442; 5,366,496; 5,397,342; 5,391,200; 5,545,202; 5,603,732; and 5,916,243, which are hereby incorporated herein by reference in their respective entireties.

Device configurations illustrated herein are generally described as capable of implementing various functions traditionally performed by an implantable cardioverter/defibrillator (ICD), and may operate in numerous cardioversion/defibrillation modes as are known in the art. Exemplary ICD circuitry, structures and functionality, aspects of which can be incorporated in an ITCS device of a type contemplated herein, are disclosed in commonly owned U.S. Pat. Nos. 6,148,230; 5,133,353; 5,179,945; 5,314,459; 5,318,597; 5,620,466; and 5,662,688, which are hereby incorporated herein by reference in their respective entireties.

An ITCS device can implement functionality traditionally provided by cardiac diagnostic devices or cardiac monitors as are known in the art. Exemplary cardiac monitoring circuitry, structures and functionality, aspects of which can be incorporated in an ITCS device of a type contemplated herein, are disclosed in commonly owned U.S. Pat. Nos. 5,313,953; 5,388,578; and 5,411,031, which are hereby incorporated herein by reference in their respective entireties.

An ITCS device may implement various anti-tachyarrhythmia therapies, such as tiered therapies, which may involve performing rate-based, pattern and rate-based, and/or morphological tachyarrhythmia discrimination analyses. Exemplary arrhythmia detection and discrimination circuitry, structures, and techniques, aspects of which can be implemented by an ITCS device of a type contemplated herein, are disclosed in commonly owned U.S. Pat. Nos. 5,301,677 and 6,438,410, which are hereby incorporated herein by reference in their respective entireties.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A medical system, comprising:
a housing configured for implantation within a patient's subclavicular region;
detection circuitry provided in the housing and configured to detect cardiac electrical activity;
energy delivery circuitry provided in the housing and configured to deliver therapy to treat a detected tachycardia or fibrillation episode; and
a lead coupled to the housing, detection circuitry, and energy delivery circuitry, the lead configured for subcutaneous, non-intrathoracic placement within the patient, the lead comprising:
a lead body configured to extend from the patient's subclavicular region to a location just below the patient's ribs or the subxiphoid process of the patient's sternum;
a defibrillation electrode provided at a distal end of the lead body; and
a pair of sensing electrodes provided on the lead body at locations that overlie a lateral aspect of the patient's left ventricle between the third and eleventh ribs.

2. The system of claim 1, wherein the pair of sensing electrodes is provided on the lead body at locations consistent with positions V2-V5 of surface electrocardiogram electrodes.

3. The system of claim 1, wherein communications circuitry is provided in the housing, the communications circuitry configured to facilitate communications between the system and a patient-external device.

4. The system of claim 3, wherein the communications circuitry is configured to facilitate communications between the system and a hand-held or bedside communications device.

5. The system of claim 3, wherein the communications circuitry is configured to facilitate communications between the system and an interface of a network.

6. The system of claim 4, wherein the network is configured to support a patient management system.

7. The system of claim 1, wherein the housing is configured for implantation in the patient's subclavian region in closer proximity to a left axillary region than a sternal region of the patient.

8. The system of claim 1, wherein the lead body is configured to extend from the patient's subclavicular region to the location just below the patient's ribs.

9. The system of claim 1, wherein the lead body is configured to extend from the patient's subclavicular region to the subxiphoid process of the patient's sternum.

10. The system of claim 1, wherein the energy delivery circuitry is configured to deliver therapy to treat the detected tachycardia or fibrillation episode using a vector defined between the housing electrode and the defibrillation electrode.

11. The system of claim 1, wherein the detection circuitry is configured to sense the cardiac electrical activity using a vector defined between the sense electrodes.

12. The system of claim 1, wherein the detection circuitry is configured to sense the cardiac electrical activity using a vector defined between at least one of the sense electrodes and the housing electrode.

13. The system of claim 1, wherein the housing electrode comprises all or a portion of an electrically conductive enclosure of the housing.

14. The system of claim 1, wherein the housing comprises a header, and the housing electrode is supported by the header.

15. The system of claim 1, wherein the housing electrode is supported by a stub lead coupled to the housing.

16. The system of claim 15, wherein the stub lead is configured to extend from the housing into the patient's left axilla and oriented posteriorly.

17. The system of claim 1, wherein the pair of sensing electrodes comprise ring electrodes.

18. The system of claim 1, wherein the defibrillation electrode comprises a coil electrode.

19. The system of claim 1, wherein the defibrillation electrode comprises a screen patch electrode.

20. A method, comprising:
providing a pulse generator disposed in a housing and implanted within a patient's subclavicular region;
providing a lead coupled to the pulse generator and configured for subcutaneous, non-intrathoracic placement within the patient, the lead comprising a lead body configured to extend from the patient's subclavicular region to a location just below the patient's ribs or the subxiphoid process of the patient's sternum, a defibrillation electrode provided at a distal end of the lead body, and a pair of sensing electrodes provided on the lead body at locations that overlie a lateral aspect of the patient's left ventricle between the third and eleventh ribs;
sensing cardiac electrical activity;
detecting a tachycardia or fibrillation episode using a vector defined between the pair of sensing electrodes;
delivering a therapy to treat the detected tachycardia or fibrillation episode using a vector defined between the housing of the pulse generator and the defibrillation electrode.

21. The method of claim 20, comprising storing information concerning one or both of the cardiac electrical activity and the tachycardia or fibrillation episode within the housing.

22. The method of claim 20, comprising communicating information concerning one or both of the cardiac electrical activity and the tachycardia or fibrillation episode to a patient-external device.

23. The method of claim 20, comprising communicating information concerning one or both of the cardiac electrical activity and the tachycardia or fibrillation episode to a network.

24. The method of claim 20, comprising communicating information concerning one or both of the cardiac electrical activity and the tachycardia or fibrillation episode to a portable communications device.

25. The method of claim 20, comprising:
communicating information concerning one or both of the cardiac electrical activity and the tachycardia or fibrillation episode to a patient-external device; and
analyzing the information to assess the patient's well-being.

26. The method of claim 20, comprising:
communicating information concerning one or both of the cardiac electrical activity and the tachycardia or fibrillation episode to a patient-external device; and
analyzing the information to implement or adjust a therapy deliverable to the patient.

27. The method of claim 20, comprising:
communicating information concerning one or both of the cardiac electrical activity and the tachycardia or fibrillation episode to a patient-external device; and
analyzing the information to generate a therapeutic recommendation for managing cardiac arrhythmia for the patient based at least in part on the analyzed information.

28. The method of claim 20, comprising:
communicating information concerning one or both of the cardiac electrical activity and the tachycardia or fibrillation episode to a patient-external device;
analyzing the information; and
generating one or both of visual and aural output based at least in part on the analyzed information.
